(19) United States
(12) Patent Application Publication
(10) Pub. No.: US 2011/0065135 A1
(43) Pub. Date: Mar. 17, 2011

(54) LIPOSOME COMPOSITION, ITS PRODUCTION PROCESS, AND METHOD FOR ANALYZING ANALYTE USING THE SAME

(76) Inventors: Daisuke Okamura, Kanagawa (JP); Ryusuke Murayama, Ehime (JP); Naoyoshi Egashira, Hiroshima (JP)

(21) Appl. No.: 12/842,663

(22) Filed: Jul. 23, 2010

(30) Foreign Application Priority Data
Jul. 27, 2009 (JP) 2009-174390

(51) Int. Cl. G01N 33/53

(52) U.S. Cl. 435/7.93; 435/7.94

(57) ABSTRACT

A liposome composition capable of including a chemical substance such as an electrochemiluminescent substance in an internal aqueous phase of the liposome at a higher concentration, and a production method thereof, as well as an analytical method of an analyte that enables an analyte to be analyzed with a high sensitivity using the liposome composition are provided. In a liposome composition containing a liposome, and a chemical substance enclosed in an internal aqueous phase of the liposome, a lipid bilayer composing the liposome has a positive or negative charge, and the chemical substance has a charge opposite to the charge of the lipid bilayer.

### TECHNICAL FIELD

The present invention relates to a liposome composition and a production method thereof, and an analytical method of an analyte using the same.

### BACKGROUND ART

According to antibody labeling techniques, a labelled antibody produced by binding a labelling substance such as an enzyme, a fluorescent substance, a chemiluminescent substance or an electrochemiluminescent substance to an antibody is allowed to react with an analyte that serves as a receptor to bind thereto. Thus, detection or quantitative determination of the analyte is enabled by detecting a signal generated from thus bound labeled-antibody in proportion to the analyte concentration.

In particular, enzyme immunoassay techniques in which an enzyme-labelled antibody prepared by labeling an antibody with an enzyme is used enables analyses to be easily carried out with a high sensitivity due to a high catalytic activity of the enzyme for the substrate. However, a problem of enzyme stability, and a problem of steric hindrance of the antigen-antibody reaction because of the enzyme having a large molecule size with respect to the antibody may occur. Accordingly, in recent years, electrochemiluminescent immunoanalytical techniques in which an antibody is labeled with an electrochemiluminescent substance such as tris(2,2′-bipyridine)ruthenium complex having a small molecule size and being comparatively stable have been put into practical applications.

However, the electrochemiluminescent immunoanalytical technique does not utilize a reaction that generates a signal as in like an enzyme reaction, but utilizes a signal generated by the labelling substance itself. Therefore, it is necessary to use a detection apparatus that is highly accurate and has a complicated optical system when an analyte at an extremely low concentration is detected.

In order to solve this problem, a method in which a liposome including a ruthenium complex that is an electrochemiluminescent substance and has been bound to a test protein (ligand) on the surface thereof is used to increase the sensitivity of the electrolytic luminescence of the ruthenium complex by way of an antigen-antibody reaction between the ligand, and a substance that executes an antigen-antibody reaction with the protein was reported (see, for example, Patent Document 1).

### PRIOR ART DOCUMENT

### Patent Document

Patent Document 1: Japanese Patent Laid-Open Publication No. 2007-101339

### SUMMARY OF THE INVENTION

### Problems to be Solved by the Invention

According to the method described in Patent Document 1, a higher level of signal sensitivity may be expected to be achieved as the liposome having a greater particle size is prepared since electrochemiluminescent substance can be included in a larger amount; however, the signal sensitivity is lowered as the liposome has a greater particle size, in effect. This results from a greater particle size of liposome accompanied by increase in the steric hindrance on the liposome surface, which leads to higher possibility of inhibition of the antigen-antibody reaction. In order to suppress this steric hindrance, liposome having a smaller particle size may be prepared; however, such size reduction is accompanied by decrease in the amount of the electrochemiluminescent substance which can be included, and thus the level of the signal generated is lowered. Namely, there is a problem of failing to result in a high level of signal when a liposome having a small pore size less likely to be affected by steric hindrance is used in an antigen-antibody reaction on the liposome surface.

The present invention solves the foregoing problems, and an object of the invention is to provide a liposome composition capable of including a chemical substance such as an electrochemiluminescent substance in an internal aqueous phase of the liposome at a higher concentration such that a high level of signal can be generated even though a liposome composition having a small particle size is used, and a production method thereof, as well as an analytical method of an analyte that enables an analyte to be analyzed with a high sensitivity using the liposome composition.

### Means for Solving the Problems

In order to solve the foregoing problems, an aspect of the present invention provides a liposome composition comprising a liposome, and a chemical substance enclosed in an internal aqueous phase of the liposome, in which a lipid bilayer composing the liposome has a positive or negative charge, and the chemical substance has a charge opposite to the charge of the lipid bilayer.

Furthermore, another aspect of the present invention provides a method for producing a liposome composition comprising the step of forming a liposome in the presence of at least one of a phospholipid and a glycolipid, a lipid bilayer-forming component that imparts a positive or negative charge to a lipid bilayer composing the liposome, a chemical substance having a charge opposite to the charge of the lipid bilayer, and water to enclose the chemical substance in the internal aqueous phase of the liposome.

Moreover, still another aspect of the present invention provides an analytical method of an analyte comprising using the liposome composition having a ligand on the external surface of the liposome to analyze the analyte by way of a binding reaction of the ligand and the analyte with a sandwich method or a competitive method, the chemical substance being capable of generating a signal.

### EFFECTS OF THE INVENTION

According to the present invention, a liposome composition capable of including a chemical substance in a liposome internal aqueous phase at a high concentration, and a production method thereof can be provided. In addition, according to the analytical method of an analyte of the present invention, a high level of a signal can be obtained even though a monolayer liposome having a small particle size is used since a liposome composition capable of including a chemical substance in the internal aqueous phase of the liposome at a high concentration is used. Thus, the method enables an analyte to be analyzed with a high sensitivity using a simple apparatus. Still further, since the lipid bilayer composing the liposome has a charge, aggregation and fusion of the liposomes can be prevented by electrostatic repulsion to provide superior storage stability, and suppression of nonspecific adsorption in antigen-antibody reaction is enabled.

### BRIEF DESCRIPTION OF THE DRAWINGS

FIG. 1 shows a schematic view illustrating a liposome composition according to the present Embodiment 1.

FIG. 2 shows a schematic view illustrating a method for producing a liposome composition according to the present Embodiment 1.

FIG. 3 shows a graph illustrating the results of measurement of the electrochemiluminescence intensity and absorbance of a tris(2,2′-bipyridyl)ruthenium complex included in the liposome in the present Embodiment 1.

FIG. 4 shows a schematic view illustrating the analytical method of an analyte carried out using the liposome composition in the present Embodiment 2.

FIG. 5 shows a graph illustrating the results of the analysis of an analyte carried out using the liposome composition in the present Embodiment 2.

FIG. 6 shows a graph illustrating the percentage of enclosure of a bisbipyridine{{4,4′-(4-aminobutyl)}-2,2′-bipyridine}ruthenium complex included in the liposome in the present Embodiment 3.

FIG. 7 shows a schematic view illustrating the analytical method of an analyte carried out using the liposome composition in the present Embodiment 4.

FIG. 8 shows a graph illustrating the results of the analysis of an analyte carried out using the liposome composition in the present Embodiment 4.

### MODE FOR CARRYING OUT THE INVENTION

Hereinafter, embodiments of the liposome composition and the production method thereof, and the analytical method of an analyte using the same of the present invention are explained in detail with reference to drawings. The “analysis” as referred to in the present invention means both the “detection” for determining the presence or absence of an analyte (target compound of the analysis), and “quantitative determination” for determining the amount of the analyte present.

### Embodiment 1

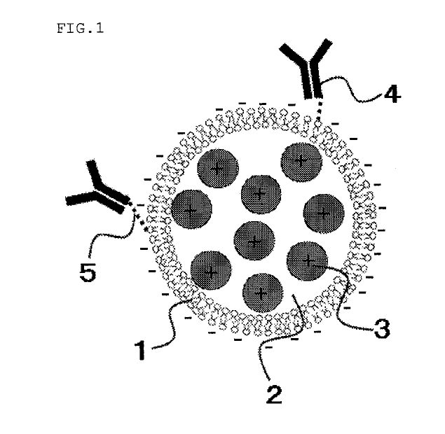

FIG. 1 schematically shows the liposome composition in Embodiment 1.

FIG. 1 depicts a lipid bilayer 1 having a negative charge, a liposome internal aqueous phase 2, a chemical substance 3 having a positive charge, a ligand 4, and a linker 5. The chemical substance 3 is present in the liposome internal aqueous phase 2, namely, is included in the liposome. The ligand 4 modifies the external surface of the liposome via the linker 5.

The lipid bilayer 1 composes the liposome in a spherical form. The component forming the lipid bilayer contains at least either one of a phospholipid or a glycolipid as a principal constitutive component. The formed liposome is a monolayer liposome having a diameter of 20 to 200 nm. The phospholipid and the glycolipid are not particularly limited, and for example, the phospholipid may include dipalmitoylphosphatidylcholine, dipalmitoylphosphatidylethanolamine, phosphatidic acid, distearoylphosphatidylcholine or the like, whereas the glycolipid may include ganglioside, galactocerebroside, glucosylcardiolipin or the like. The carbon chain of a fatty acid in these phospholipid and glycolipid preferably has 12 to 18 carbon atoms.

Although the lipid bilayer 1 has, a negative charge, this negative charge is imparted to the lipid bilayer by containing a component having a negative charge in the component that forms the lipid bilayer. Examples of such a component that imparts a negative charge to the lipid bilayer include cardiolipin and sulfatide, acidic phospholipids such as phosphatidylglycerol and phosphatidylserine, glycolipids having a sialic acid, phosphoric acid esters such as dicetyl phosphate, and the like. Among the phosphoric acid esters, phosphoric acid diesters are particularly preferred, and the hydrocarbon group contained in the phosphoric acid ester preferably has 12 to 18 carbon atoms. The lipid bilayer will have a negative charge by allowing such a component to be contained in the lipid bilayer-forming component.

In order to improve the stability of the liposome, cholesterol may be contained in the aforementioned lipid bilayer-forming component such as the phospholipid or glycolipid as needed. The amount of the blended cholesterol in this case may be adjusted at a rate of 10 to 500% by mole relative to the lipid bilayer-forming component. In addition, for the purpose of preventing oxidization of the lipid, α-tocopherol may be also contained, and in this instance, the amount of blending may be adjusted appropriately depending on the characteristics of the liposome.

The liposome internal aqueous phase 2 is an aqueous phase present inside a spherical lipid bilayer that composes the liposome, and contains the chemical substance 3 herein. The internal aqueous phase is composed of a water-based solvent, and the chemical substance is dissolved in the water-based solvent. Thus, the liposome composition of the present invention can include the chemical substance in the internal aqueous phase of the liposome.

The chemical substance 3 is a compound having a positive charge. In Embodiment 1, a metal complex having an electrochemiluminescent property and hydrophilicity was used as the chemical substance 3. However, the chemical substance 3 is not particularly limited as long as it is dissolved in a hydrophilic solvent and is a compound having a positive charge.

The metal complex having an electrochemiluminescent property refers to a metal complex capable of emitting light by applying a voltage to the solution containing the same. The metal complex consists of a central metal and a ligand. The central metal is not particularly limited as long as it has an electrochemiluminescent property. For example, ruthenium, osmium, chromium, copper, iridium, rhenium, europium, or the like may be included. Among these, ruthenium or osmium is preferred since high light emission efficiency is achieved.

The ligand is not particularly limited as long as the metal complex containing the same has an electrochemiluminescent property; however, a organic compound containing at least one functional group selected from the group consisting of an amino group, a phosphino group, a carboxyl group and a thiol group, or a heterocyclic compound is suitably employed. Both the aforementioned organic compound and the heterocyclic compound may be incorporated as the ligand. The heterocyclic compound is suitably a compound having a pyridine moiety in light of the efficiency of light emission, and most suitably a compound having a bipyridine skeleton or phenanthroline skeleton. The compound having a bipyridine skeleton or phenanthroline skeleton may be bipyridine or phenanthroline not having a substituent, or bipyridine or phenanthroline having a substituent such as phenyl. As the compound having a bipyridine skeleton, 2,2′,2″-terpyridine may be used. In addition, 8-hydroxyquinoline, 2-phenylpyridine or the like may be also used. As the aforementioned organic compound, an organic compound containing a phosphino group is preferred in light of the efficiency of light emission, and bis(diphenylphosphino)ethene is particularly preferred. In Embodiment 1, a tris(2,2′-bipyridyl)ruthenium complex is used as the metal complex having an electrochemiluminescent property and hydrophilicity, but not limited thereto according to the present invention.

Inclusion of the chemical substance 3 into the liposome internal aqueous phase 2 may be realized by adding the chemical substance to the water-based solvent used in forming the liposome to dissolve therein. In Embodiment 1, a positively charged hydrophilic chemical substance is used as the chemical substance that imparts a negative charge to the liposome lipid bilayer and is included in the liposome. Accordingly, the influence of an electrostatic action between the lipid bilayer and the chemical substance enables the chemical substance 3 to be included in the liposome internal aqueous phase 2 with a higher concentration.

In Embodiment 1, a liposome composition is explained in which a chemical substance having a positive charge is included in the internal aqueous phase of the liposome composed of the lipid bilayer to which a negative charge was imparted. However, also a liposome in which a chemical substance having a negative charge is included in the internal aqueous phase of the liposome composed of the lipid bilayer to which a positive charge was imparted can similarly include the chemical substance in the liposome internal aqueous phase at a higher concentration. In this case, a positive charge can be imparted to the lipid bilayer by containing the lipid bilayer-forming component having a positive charge in the lipid bilayer-forming component of the liposome. As such a component, a component capable of imparting a positive charge to the lipid bilayer whereas being a component that forms the lipid bilayer is acceptable. For example, an amine compound such as stearylamine may be included, but not limited thereto. Among the amine compounds, a primary amine is preferred, and the amine compound preferably has a hydrocarbon group of 12 to 18 carbon atoms.

The ligand 4 is positioned on the external surface side of the liposome, and bound to the lipid bilayer 1 via the linker 5. The ligand 4 may contain at least one selected from the group consisting of an antibody, an antigen, a peptide, a nucleic acid, protein A, protein G, avidin, and biotin. In the present Embodiment 1, streptavidin which is one type of avidin is used as the ligand.

The linker 5 is a component for allowing the ligand 4 to bind to the external surface of the lipid bilayer 1. Specifically, by binding to both the lipid bilayer-forming component and the ligand 4, binding of the ligand 4 to the external surface of the lipid bilayer 1 is permitted. In the present Embodiment 1, SPDP-DPPE (hereinafter, may be abbreviated as active DPPE) is prepared by allowing N-succinimidyl 3-(2-pyridyldithio)propionate (hereinafter, may be abbreviated as SPDP) to bind to the amino group of dipalmitoylphosphatidylethanolamine (hereinafter, may be abbreviated as DPPE) via a condensation reaction, and a chemical substance-including liposome is prepared using this active DPPE as one liposome lipid bilayer-forming component. On the other hand, SPDP-modified streptavidin is prepared by allowing SPDP to bind to the amino group of streptavidin via a condensation reaction. By permitting disulfide binding between the active DPPE present on the liposome lipid bilayer and SPDP-modified streptavidin, streptavidin modifies the external surface of liposome. However, the modification of a liposome surface with a ligand of the present invention is not limited thereto.

Next, the method for producing a liposome composition of the present invention is described.

In the present invention, the liposome may be formed, by any commonly known method such as, for example, vortexing method, sonication method, prevesicle method, ethanol infusion method, French press method, extrusion method, or the like. Specifically, the aforementioned method for forming a liposome is first carried out in the presence of at least one of a phospholipid and a glycolipid, a lipid bilayer-forming component that imparts a positive or negative charge to a liposome lipid bilayer, a chemical substance having a charge opposite to the charge of the liposome lipid bilayer, water, and a lipid bilayer-forming component to which a linker is bound as needed to enable a liposome composition having the chemical substance included in the liposome internal aqueous phase to be obtained. More specifically, the liposome composition is formed according to the method described above by mixing at least one of a phospholipid and a glycolipid, and a lipid bilayer-forming component that imparts a positive or negative charge to a liposome lipid bilayer with an aqueous solution dissolving a chemical substance having a charge opposite to the charge of the lipid bilayer-forming component to be included in the liposome internal aqueous phase.

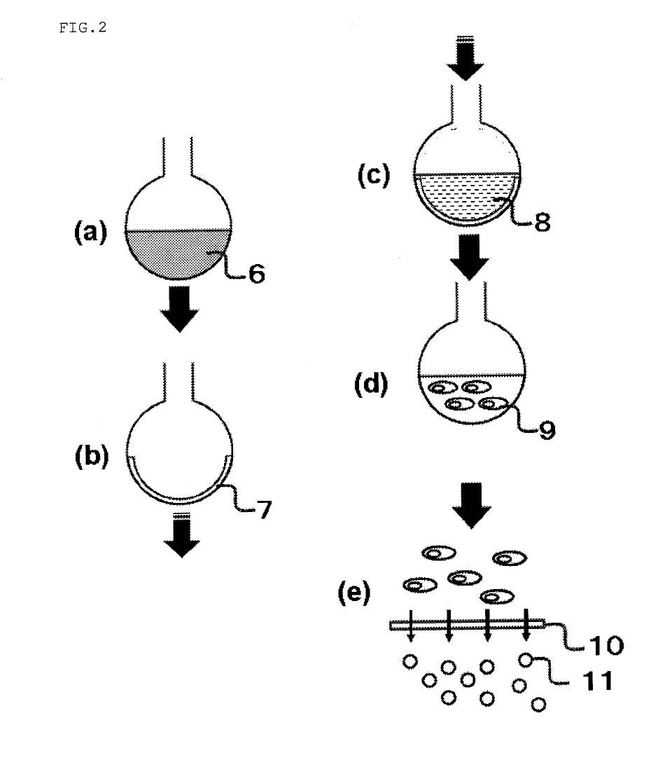

In the present Embodiment 1, the liposome composition of the present invention is prepared with an extrusion method. FIG. 2 shows one example of the method for producing a liposome composition of the present Embodiment 1, but the present invention is not limited thereto.

FIG. 2 depicts a lipid bilayer-forming component 6 having a negative charge dissolved in a solvent including a nonpolar solvent, a lipid film 7, an aqueous solution 8 dissolving a chemical substance having a positive charge, a multilayer liposome composition 9, a filter 10, and a monolayer and uniform liposome composition 11.

First, a lipid bilayer-forming component mixture containing at least a lipid bilayer-forming component that imparts a negative charge to the liposome lipid bilayer and at least one of a phospholipid and a glycolipid is dissolved in a solvent including a nonpolar solvent, and charged in a glass vessel such as a recovery flask (FIG. 2 (a)). As the nonpolar solvent, for example, chloroform, dichloromethane, dichloroethane, dichloroethylene, tetrachloroethane, carbon tetrachloride, trichloroethylene, ether, tetrahydrofuran, hexane, cyclohexane or the like may be used. Since dissolution of the lipid is facilitated, an alcohol such as methanol may be also contained in the nonpolar solvent.

Next, the nonpolar solvent is eliminated by evaporation or under a flow of gas such as nitrogen to form a thin film of the lipid (lipid film) on the wall face of the glass vessel (FIG. 2 (b)). To thus formed thin film of the lipid, an aqueous solution dissolving a chemical substance having a positive charge is added to allow the thin film to be swollen (FIG. 2 (c)). Then, a multilayer liposome composition including a chemical substance having a positive charge is formed by applying mechanical vibration to the glass vessel (FIG. 2 (d)). As the mechanical vibration in this step, vigorous stirring, ultrasonic vibration or the like may be employed, and thus formation of a multilayer liposome composition is enabled. In Embodiment 1, a tris(2,2′-bipyridyl)ruthenium complex is used as the chemical substance having a positive charge.

Next, the formed multilayer liposome composition is passed through a filter to enable to have a uniform particle size, whereby a liposome composition having a monolayer and uniform particle size is formed (FIG. 2 (e)). In Embodiment 1, an extrusion method is applied for homogenizing the liposome composition.

According to the method for producing a liposome composition of the present invention, similar effects can be achieved even though carried out using a lipid bilayer-forming component mixture containing a lipid bilayer-forming component that imparts a positive charge to the liposome lipid bilayer, and using a chemical substance having a negative charge as a chemical substance to be included in the liposome internal aqueous phase.

Hereinafter, Examples of method for producing a liposome composition are described.

<Preparation of Active DPPE>

Preparation of active DPPE was carried out according to the reaction scheme represented by the chemical formula 1.

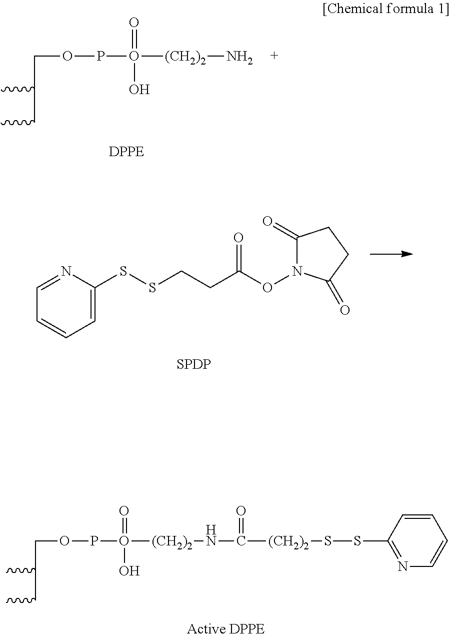

DPPE in an amount of 10 μmol, 12 μmol of SPDP, and 20 μmol of triethylamine (TEA) were dissolved in a solution of chloroform/anhydrous methanol=9:1, and the mixture was stirred at room temperature for 2 hrs. After the stirring for 2 hrs, a 10 mmol/l phosphate buffer (pH 7.4) containing potassium dihydrogen phosphate was added thereto. The mixture was vigorously stirred, and the top phase was removed. Thereto was added distilled water then, and the mixture was vigorously stirred. The top phase was removed, and the bottom phase was collected. The solvent of the collected bottom phase was distilled away, and dried under reduced pressure for 12 hrs to obtain active DPPE in a recovery amount of 5.8 mg (6.52×10⁻⁶ mol), at a yield of 65.24%.

<Preparation of Liposome Composition>

In a recovery flask, dipalmitoylphosphatidylcholine (hereinafter, may be abbreviated as DPPC), active DPPE, cholesterol, and dicetyl phosphate respectively dissolved in a solution prepared to give a ratio of chloroform/anhydrous methanol=9:1 were placed, and the solvent was distilled away with an evaporator to form a lipid film on the inside wall of the flask. The amounts of charged DPPC, active DPPE, cholesterol and dicetyl phosphate were adjusted as shown in Table 1.

TABLE 1

| Liposome constitutive | Condition | Condition | Condition | Condition | Condition | Condition |
|---|---|---|---|---|---|---|
| component | 1 | 2 | 3 | 4 | 5 | 6 |
| DPPC (μmol) | 12.63 | 10 | 13.3 | 16.67 | 12.63 | 10 |
| Active DPPE (μmol) | 0.03 | 0.03 | 0.03 | 0.03 | 0.03 | 0.03 |
| Cholesterol (μmol) | 10 | 10 | 10 | 10 | 10 | 10 |
| Dicetyl phosphate | 0.67 | 6.67 | — | — | — | — |
| (μmol) | | | | | | |
| Stearylamine (μmol) | — | — | — | — | 0.67 | 6.77 |
| Total (μmol) | 23.33 | 26.7 | 23.33 | 26.7 | 23.33 | 26.7 |

In Table 1, conditions 1 and 2 demonstrate those for preparing the liposome compositions of the present invention. On the other hand, in order to confirm the effects of the present invention, conditions 3 and 4 demonstrate those for preparing control liposome compositions formed with DPPC, active DPPE and cholesterol without imparting a charge to the liposome lipid bilayer, whereas conditions 5 and 6 demonstrate those for preparing the liposome compositions formed from DPPC, active DPPE, cholesterol and stearylamine, to which a positive charge was imparted to the liposome lipid bilayer.

After the lipid film formed as described above was dried under reduced pressure, 1 ml of an aqueous solution dissolving a 5 mmol/l tris(2,2′-bipyridyl)ruthenium complex as a chemical substance was injected. Thereafter, the recovery flask having the lipid film formed therein was vigorously stirred to allow a multilayer liposome composition to be formed. Thus prepared multilayer liposome composition was subjected to sizing by passing through a polycarbonate membrane having a pore size of 100 nm with an extrusion method to impart a negative charge to the lipid bilayer. Accordingly, a uniform and monolayer liposome composition that includes a tris(2,2′-bipyridyl)ruthenium complex was prepared. Furthermore, purification with a gel filtration column (Sephadex G-50), and fractionation were carried out. Thus obtained chemical substance-including liposome fraction was measured on a microplate reader (manufactured by Molecular Devices, Inc.) at an excitation wavelength of 444 nm and a fluorescent wavelength of 612 nm, and fractions identified to exhibit fluorescence were collected. The fraction containing the liposome composition prepared under each condition was diluted to 15 ml in total. The particle size of the prepared liposome composition was determined using “Zetasizer Nano” available from Sysmex Corporation (manufactured by Malvern Instruments Ltd) with a dynamic light scattering method. The conditions carrying out the particle size measurement involved: a measurement temperature of 25° C.; a diluent of a 10 mmol/l phosphate buffer (pH 7.4) consisting of potassium dihydrogen phosphate; and a measurement mode of Auto. In addition, analyses were performed with the setting of the refractive indices of the liposome and the diluent being 1.45 and 1.33, respectively, in the dynamic light scattering method. From the particle grade distribution derived from the measurement results, the mean particle diameter was determined. The results of the determined mean particle diameter are shown in Table 2.

| | Condition | Condition | Condition | Condition | Condition | Condition |
|---|---|---|---|---|---|---|
| | 1 | 2 | 3 | 4 | 5 | 6 |
| Average of liposome | 97.9 | 99.7 | 101 | 100 | — | — |
| particle size (nm) | | | | | | |

As a result of the measurement of the particle size with the dynamic light scattering method, the mean particle diameter of the liposome composition was: 97.9 nm under the condition 1, 99.7 nm under the condition 2, 101 nm under the condition 3, and 100 nm under the condition 4. Thus, the particle size of the liposome composition prepared under the conditions 1-4 was approximately the same. Therefore, it is believed that the solutions containing thus prepared liposome composition contained almost the same number of liposomes between the condition 1 and the condition 3, and the condition 2 and the condition 4, as the same molar quantities of the lipid bilayer-forming component were added. On the other hand, in the case of the condition 5 and the condition 6 in which stearylamine was added for imparting a positive charge to the liposome lipid bilayer, confirmation of liposome formation failed. The cause of this result is believed to be electrostatic repulsion that occurred in the step of forming the lipid bilayer between the tris(2,2′-bipyridyl)ruthenium complex having a positive charge, and the lipid bilayer-forming component having the identical positive charge, which lead to failure in liposome formation.

<Preparation of SPDP-Modified Ligand>

Next, preparation of the SPDP-modified ligand was carried out according to the reaction scheme represented by the chemical formula 2.

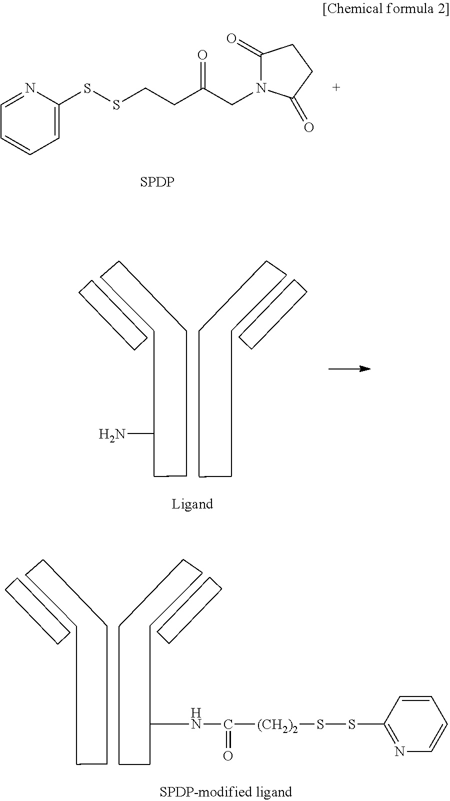

A 1.6 mmol/l SPDP solution dissolved in anhydrous methanol in an amount of 5 μl was added to 1 mg/ml streptavidin (ligand), and the reaction was allowed at room temperature for 30 min. Next, thus obtained reaction liquid was placed into a dialysis membrane tube having a fractionation molecular weight of 1 kDa, and dialysis for a 10 mmol/l acetate buffer (pH 4.5) was carried out for 3 days to recover SPDP-modified streptavidin (SPDP-modified ligand).

<Modification of Liposome Composition Surface with Ligand>

Moreover, modification of the surface of the liposome composition with a ligand was carried out according to the reaction schemes represented by the chemical formula 3 and the chemical formula 4.

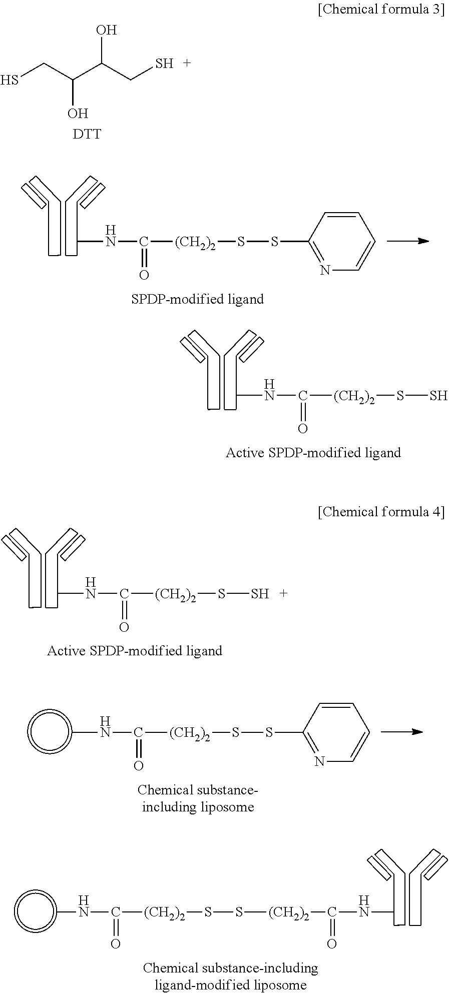

A 50 mmol/l dithiothreitol (hereinafter, may be abbreviated as DTT) dissolved in a 10 mmol/l acetate buffer (pH 4.5) in an amount of 500 μl was added to the dialyzed SPDP-modified streptavidin, and the reaction was allowed at room temperature for 30 min. The SPDP-modified streptavidin after completing the reaction was fractionated on Sephadex G25 to remove DTT, and pyridine-2-thione generated as a by-product. Thus, a fraction containing active SPDP-modified streptavidin was obtained. The fraction of active SPDP-modified streptavidin was added to 10 ml of a chemical substance-including liposome solution, and the reaction was allowed at room temperature for 24 hrs. After completing the reaction, fractionation on Shepharose 4B at 4° C. was carried out to remove unbound SPDP-modified streptavidin. Thereafter, fluorescence was measured at an excitation wavelength of 444 nm and a fluorescent wavelength of 612 nm, and fractions identified to exhibit fluorescence were collected. Accordingly, a solution containing a liposome composition modified with the ligand on the surface thereof was obtained.

<Measurement of Tris(2,2′-Bipyridyl)ruthenium Complex in Liposome Composition>

First, the solution containing the prepared liposome composition was diluted 400 times with a 10 mmol/l phosphate buffer (pH 7.4), and the electrochemiluminescence intensity was measured. On a glass substrate, 200 nm of gold was formed with 10 nm of titanium as a base using a sputtering apparatus (manufactured by ULVAC, Inc., SH-350) to form an electrode pattern by a photolithography process. To thus obtained gold electrode chip having a working electrode having a diameter of 3 mm, 2 μl of the solution containing the liposome composition which had been diluted 400 times was dropped on the working electrode, and left to stand in a 65° C. thermoregulated bath for 5 min. Thereafter, 80 μl of an electrolytic luminescence liquid containing 0.1 mol/l potassium dihydrogen phosphate, 0.1 mol/l dipotassium hydrogen phosphate and 0.1 mol/l TEA was added dropwise, and voltage scanning from 0 V to 1.3 V was carried out to determine an electrochemiluminescence intensity for 4 sec with a photomultiplier tube (manufactured by Hamamatsu Photonics K.K., H7360-01). Quantitative determination of the liposome concentration was carried out based on a calibration curve produced beforehand in a similar manner with the tris(2,2′-bipyridyl)ruthenium complex. The results of measurement of the electrochemiluminescence intensity of the chemical substance-including liposome are shown in Table 3.

TABLE 3

| | Condition 1 | Condition 2 | Condition 3 | Condition 4 |
|---|---|---|---|---|
| Electrochemiluminescence | 25,468 | 45,221 | 18,816 | 19,744 |
| intensity (RLU) | | | | |

The electrochemiluminescence intensity (integrated value) of the liposome compositions prepared under the condition 1 to condition 4 was: 25,468 RLU under condition 1; 45,221 RLU under condition 2; 18,816 RLU under condition 3; and 19,744 RLU under condition 4. The unit RLU representing the electrochemiluminescence intensity is an abbreviation indicating “Relative Light Unit”.

On the basis of these results, determination of the concentration from the calibration curve of the tris(2,2′-bipyridyl)ruthenium complex produced with the concentrations of 62.5 nmol/l, 31.3 nmol/l, 15.6 nmol/l, 7.8 nmol/l, 3.9 nmol/l and 0 revealed that the concentration of the tris(2,2′-bipyridyl)ruthenium complex contained in the chemical substance-including liposome diluted to 15 ml in total was: 6.2 μmol/l under the condition 1; 12.3 μmol/l under the condition 2; 4.2 μmol/l under the condition 3; and 4.5 μmol/l under the condition 4. Since the particle size of the liposome prepared in the conditions 1 and 3, and the conditions 2 and 4 was approximately the same, the number of the liposomes contained in the solution was believed to be almost the same. Therefore, it can be concluded that the volume of the entire internal aqueous phase of the liposome contained in the solution was nearly equal. It was thus suggested that the chemical substance can be enclosed in the liposome at a high concentration according to the present invention by determining the percentage of enclosure of the tris(2,2′-bipyridyl)ruthenium complex based on the above procedure. The percentage of enclosure of the tris(2,2′-bipyridyl)ruthenium complex was calculated according to the following formula.

Y=X/Z×100

Y: percentage of enclosure (%) of the tris(2,2′-bipyridyl)ruthenium complex
X: concentration (mol/l) of the tris(2,2′-bipyridyl)ruthenium complex enclosed in the entire liposome contained in the solution
Z: concentration (mol/l) of the tris(2,2′-bipyridyl)ruthenium complex introduced before enclosing

As a result, since the concentration of the tris(2,2′-bipyridyl)ruthenium complex introduced before enclosing was 5 mmol/l, the percentage of enclosure was 0.124% under the condition 1, 0.246% under the condition 2, 0.084% under the condition 3, and 0.09% under the condition 4. Therefore, it is proven that the tris(2,2′-bipyridyl)ruthenium complex was contained in the liposome internal aqueous phase: at 47% relative to the conventional method, under the condition 1 in which dicetyl phosphate in an amount of 3% by mole relative to the total liposome lipid bilayer-forming component was added; and at a high concentration of 173% relative to the conventional method, under the condition 2 in which dicetyl phosphate in an amount of 22% by mole relative to the total liposome lipid bilayer-forming component was added. Still further, in order to verify the concentration of the tris(2,2′-bipyridyl)ruthenium complex in the liposome composition, the absorbance of the chemical substance-including liposome solution diluted to 15 ml in total was measured at a wavelength of 450 nm with a microplate reader. The results of the measurement are shown in Table 4.

TABLE 4

| | Condition 1 | Condition 2 | Condition 3 | Condition 4 |
|---|---|---|---|---|
| Absorbance | 0.223 | 0.419 | 0.163 | 0.184 |
| (450 nm) | | | | |

As a result of the measurement of the absorbance on the sample prepared under conditions 1 to 4, respectively, the absorption was 0.223 under the condition 1, 0.419 under the condition 2, 0.163 under the condition 3, and 0.184 under the condition 4. Determination of each concentration based on the calibration curve produced beforehand for the tris(2,2′-bipyridyl)ruthenium complex with the concentrations of 66.7 μmol/l, 33.3 μmol/l, 16.7 μmol/l, 8.3 μmol/l, 4.2 μmol/l and 0 revealed that the concentration of the tris(2,2′-bipyridyl)ruthenium complex contained in the liposome internal aqueous phase was: 22.18 μmol/l under the condition 1; 46.69 μmol/l under the condition 2; 14.69 μmol/l under the condition 3; and 17.31 μmol/l under the condition 4. When the percentage of enclosure of the tris(2,2′-bipyridyl)ruthenium complex was determined, it was 0.444% under the condition 1, 0.934% under the condition 2, 0.294% under the condition 3, and 0.346% under the condition 4. Therefore, it is consequently proven that the tris(2,2′-bipyridyl)ruthenium complex was contained in the liposome internal aqueous phase: at 51% relative to the conventional method, under the condition 1 in which dicetyl phosphate in an amount of 3% by mole relative to the total liposome lipid bilayer-forming component was added; and at a high concentration of 170% relative to the conventional method, under the condition 2 in which dicetyl phosphate in an amount of 22% by mole relative to the total liposome lipid bilayer-forming component was added.

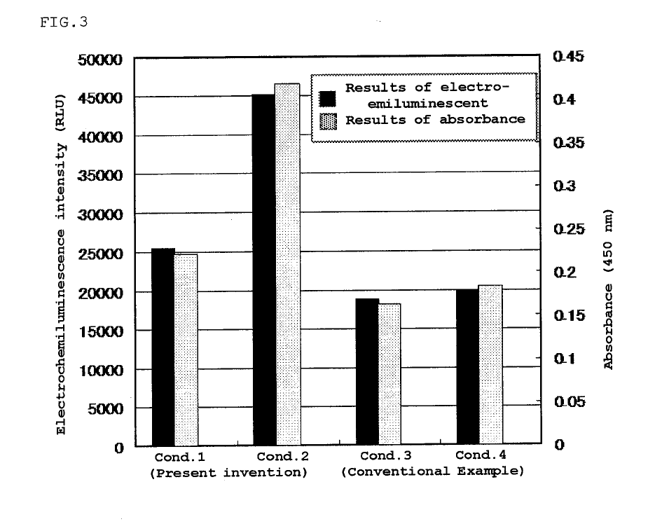

The foregoing results are shown in FIG. 3. As is seen from FIG. 3, the results of the measurement of the electrochemiluminescence intensity and the absorbance exhibited a consistent tendency. Both data reveal that by using the liposome composition of the present invention and production method thereof, addition of 3% by mole of dicetyl phosphate increased the amount of included metal complex by about 50%, whereas addition of 22% by mole of dicetyl phosphate increased the amount of included metal complex by about 170%, as compared with conventional liposome compositions. In other words, use of the liposome composition of the present invention and production method thereof enables a chemical substance having a positive charge to be included in the liposome internal aqueous phase at a high concentration.

In Embodiment 1, one example of allowing a chemical substance having a positive charge to be included in a liposome having a negative charge, but a similar effect can be obtained also when a chemical substance having a negative charge is included in a liposome having a positive charge. This can be easily inferred from the results that indicated inclusion at a high concentration of the chemical substance in the liposome internal aqueous phase of the present Embodiment 1 due to an electrostatic action, and the results of failure in formation of the liposome due to the influence of the electrostatic repulsion in an attempt to allow inclusion of a chemical substance having a positive charge in a liposome having a positive charge.

### Embodiment 2

Embodiment 2 concerns an analytical method of an analyte in which a liposome composition is used. Upon analyses of analytes, similar effects can be obtained even though any one of a noncompetitive method (sandwich method) and a competitive method which is a general immunoanalytical technique is employed. However, a noncompetitive method (sandwich method) in which magnetic beads are used is explained as one example in Embodiment 2.

In the chemical substance-including ligand-modified liposome, the analyte and the ligand may bind either directly or indirectly, and a similar effect can be achieved in either case. In the present Embodiment, the analysis of the analyte is carried out by allowing the liposome composition modified with streptavidin according to Embodiment 1 to bind to a complex in which the analyte binds to a biotin labelled antibody, thereby permitting indirect binding of the analyte to the streptavidin-modified liposome composition.

In Embodiment 2, the analysis of the analyte is carried out by electrochemiluminescence emitted from the tris(2,2′-bipyridyl)ruthenium complex which is a chemical substance having a positive charge. However, in the case of the chemical substance having a fluorescence property such as the tris(2,2′-bipyridyl)ruthenium complex, similar effects can be achieved also when an analysis is carried out on the analyte by detection of the fluorescence in addition to the electrochemiluminescence.

Hereinafter, an immunoassay technique is specifically described.

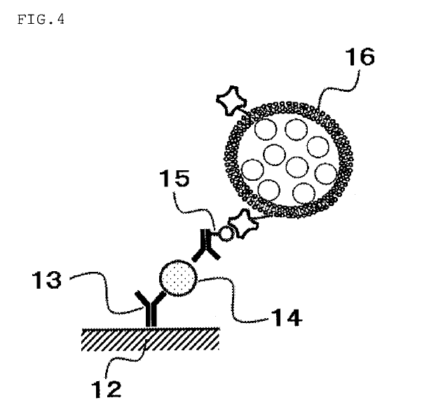

The analytical method of an analyte according to the present Embodiment 2 is carried out based on the reaction shown in FIG. 4. FIG. 4 depicts a solid phase 12, an antibody 13 specifically binds to an analyte 14, the analyte 14, a compound 15 specifically binds to the analyte 14 and a ligand, and a liposome composition 16 modified with the ligand.

As the solid phase 12, magnetic beads are used in Examples of Embodiment 2. However, the solid phase is not particularly limited, and the analyte can be analyzed also using, for example, a gold electrode on which a self-assembled monomolecular film is formed to immobilize an antibody 13 on the gold electrode.

The analyte 14 is at least one selected from the group consisting of an antibody, an antigen, a peptide, and a nucleic acid.

The antibody 13 is not particularly limited as long as it is immobilized on the solid phase 12, and capable of specifically binding to the analyte 14.

Although the compound 15 that specifically binds to the analyte 14 and the ligand is not particularly limited, a biotin labelled antibody that binds to streptavidin which is a ligand for modifying the liposome is used in Examples of Embodiment 2. However, the liposome may be modified with a linker capable of directly and specifically binding to the analyte 14 with use of the compound 15 omitted.

The ligand for modifying the liposome composition 16 is not particularly limited as long as it can specifically bind to the compound 15 or the analyte 14. As such a ligand include, for example, an antibody, an antigen, a peptide, a nucleic acid, protein A, protein G, (strept)avidin, biotin, and the like may be exemplified.

Hereinafter, Examples in connection with Embodiment 2 are demonstrated. In the following Examples, mouse anti-human TNF-α was used as the analyte 13, and Human TNF-α (hereinafter, may be abbreviated as TNF-α) was used as the analyte 14.

Magnetic beads (manufactured by Invitrogen, Dynabeads M-450 Tosyl activated) in an amount of 25 μl (1×10⁷ beads) were collected, and washed twice with a 0.1 mol/l phosphate buffer (pH 7.4), followed by addition of 100 μg of mouse anti-human TNF-α manufactured by R&D systems, Inc. The reaction was allowed for 24 hrs, and mouse anti-human TNF-α was immobilized on the magnetic beads. Thereafter, the beads were washed with 0.1 mol/l phosphate buffered physiological saline (pH 7.4) containing 0.2 mol/l EDTA and 0.1% BSA, and then a 0.2 mol/l Tris buffer (pH 8.5) containing 0.1% BSA. Thereafter, the surface of the magnetic beads were blocked with 0.1 mol/l phosphate buffered physiological saline (pH 7.4) containing 1% BSA to prepare antibody-immobilized magnetic beads. In the following immunoassay, 5×10⁵ beads were used per assay.

Recombinant Human TNF-α manufactured by R&D systems, Inc., as an analyte was diluted with 0.1 mol/l phosphate buffered physiological saline (pH 7.4) containing 1% BSA to prepare assay sample solutions each having a concentration of 100 pg/ml, 10 pg/ml, 1 pg/ml, 0.1 pg/ml, or 0 (background: BG). A 500-μl aliquot of the sample solution was added to the antibody-immobilized magnetic beads, and the antigen-antibody reaction was allowed at 25° C. for 1 hour. After completing the antigen-antibody reaction, the beads were washed three times with 0.1 mol/l phosphate buffered physiological saline (pH 7.4).

Thereafter, 500 μl of 1 μg/ml biotin labelled antibody (biotinylated goat anti-human TNF-α) was added thereto, and the antigen-antibody reaction was allowed at 25° C. for 1 hour. After completing the antigen-antibody reaction, the beads were washed three times with 0.1 mol/l phosphate buffered physiological saline (pH 7.4).

After washing, 1 ml of the liposome composition modified with streptavidin which had been prepared under the condition 2 in Example of Embodiment 1 was added, and the reaction was allowed at 25° C. for 1 hour. After washing three times with 0.1 mol/l phosphate buffered physiological saline (pH 7.4), the beads were suspended in 20 μl of a 0.1 mol/l phosphate buffer (pH 7.4).

After the suspension was obtained, a 2.5-μl aliquot was collected, which was dropped on a working electrode of a gold electrode chip provided with the working electrode having a diameter of 3 mm on which an electrode pattern had been formed by a photolithography process on a glass substrate by forming a 200 nm of gold using a sputtering apparatus (manufactured by ULVAC, Inc., SH-350) with 10 nm of titanium as a base. After the dropping, the sample was left to stand in a 65° C. thermoregulated bath for 5 min. Thereafter, 80 μl of a liquid for electrolytic luminescence containing 0.1 mol/l potassium dihydrogen phosphate, 0.1 mol/l dipotassium hydrogen phosphate and 0.1M TEA was dropped, and subjected to voltage scanning from 0 V to 1.3 V. Thus, the electrochemiluminescence intensity was measured with a photomultiplier tube (manufactured by Hamamatsu Photonics K.K., H7360-01) for 4 sec.

As Conventional Example, the liposome composition modified with streptavidin prepared under the condition 4 in Example of Embodiment 1 was subjected to the measurement in a similar procedure.

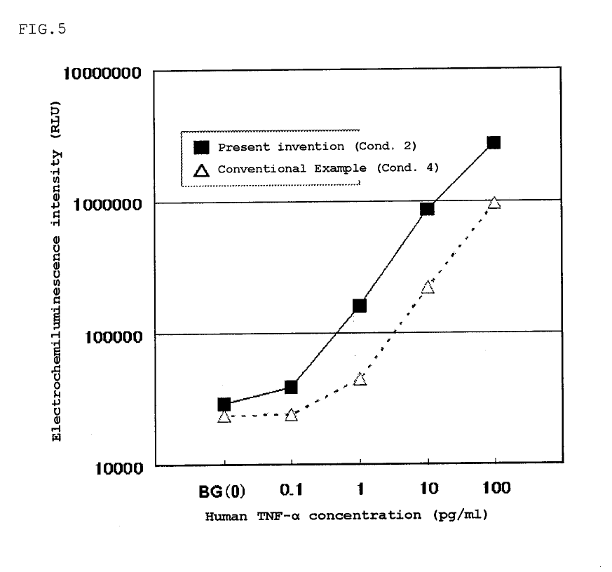

The above results are shown in FIG. 5. The electrochemiluminescence intensity according to the present invention (condition 2) was 2,761,223 RLU (100 pg/ml), 869,142 RLU (10 pg/ml), 162,336 RLU (1 pg/ml), 39,233 RLU (0.1 pg/ml), and 26,133 RLU (BG). Whereas, the electrochemiluminescence intensity according to the conventional method (condition 4) was 971,129 RLU (100 pg/ml), 226,111 RLU (10 pg/ml), 45,122 RLU (1 pg/ml), 24,226 RLU (0.1 pg/ml), and 23,817 RLU (BG). Although the electrochemiluminescence intensity was elevated 3 to 4 times in the present invention as compared with the conventional method, the background did not significantly differ between two test lines. In addition, contrary to elevation of the electrochemiluminescence intensity found around 0.1 to 1 pg/ml according to the conventional method, elevation relative to the background was found at 0.1 pg/ml in the present invention, revealing increase in the sensitivity by approximately one order of magnitude.

From the foregoing results, it was proven that the analytical method of an analyte in which the liposome composition of the present invention was used enabled three to four times the electrochemiluminescence intensity to be achieved as compared with the conventional method. Therefore, it is suggested that analyses of an analyte are enabled with high sensitivity even though a more convenient luminescence detection apparatus is used.

### Embodiment 3

In Embodiment 3, a liposome including a ruthenium complex was prepared in a similar procedure to Embodiment 1, and the liposome surface was modified with an BSA antibody derived from rabbit. Thus, the percentage of enclosure of the chemical substance included in the liposome was decided with a measuring method different from that in Embodiment 1. The reason for deciding the percentage of enclosure Embodiment 1 with a measuring method different from that in Embodiment 1 is as in the following. Liposomes have an extremely small size, and accurate determination of the number of the prepared liposome, the volume of the liposome internal aqueous phase, and the like is difficult. Therefore, diversified study for elucidating prospection for improvement of the percentage of enclosure of liposomes according to the present invention by deciding the percentage of enclosure using the measuring method from other point of view would be preferable.

The method for preparing a liposome is different from that in Embodiment 1 in terms of sizing of the prepared liposome composition with the extrusion method carried out using a polycarbonate membrane having a pore size of 50 nm, and preparation of the liposome with the liposome constitutive component containing 10 μmol of DPPC, 5 μmol of cholesterol and 1.5 μmol of active DPPE, to which 0, 1, 2, 4, 6, or 8 μmol of dicetyl phosphate was added. Moreover, the present Embodiment is different from Embodiment 1 also in terms of formation of the multilayer liposome carried out with 1 ml of an aqueous solution dissolving 1.5 μmol/l of a bisbipyridine{{4,4′-(4-aminobutyl)}-2,2′-bipyridine}ruthenium complex represented by the following formula added in place of the tris(2,2′-bipyridyl)ruthenium complex. Modification of the liposome surface with an antibody is similarly carried out to Embodiment 1 except that the TNF-α antibody was changed to a BSA antibody. With respect to other aspects, preparation was carried out in a procedure similar to that in Embodiment 1. The particle size of the liposome was measured using similarly to Embodiment 1, using “Zetasizer Nano” available from Sysmex Corporation (manufactured by Malvern Instruments Ltd) with a dynamic light scattering method. Thus, it was confirmed that any of the liposome has a mean particle diameter of about 90 nm.

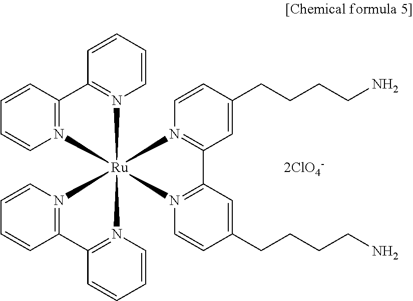

The percentage of enclosure in the prepared liposome including the bisbipyridine{{4,4′-(4-aminobutyl)}-2,2′-bipyridine}ruthenium complex was determined by detecting the ruthenium complex used in preparing this liposome, and the ruthenium complex included in this liposome with HPLC, and calculating the area of peak corresponding to each complex. This liposome was detected with a detector for the absorbance at a wavelength of 450 nm on HPLC.

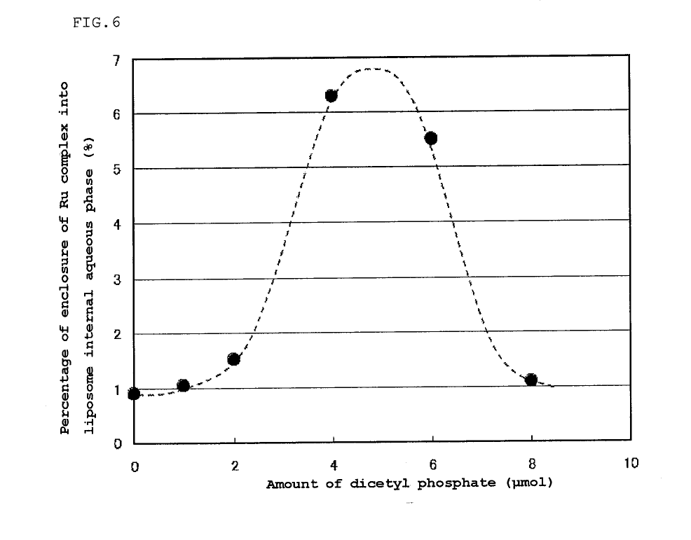

The measurement results are shown in FIG. 6. As is seen from FIG. 6, as the amount of dicetyl phosphate increased, the percentage of enclosed bisbipyridine{{4,4′-(4-aminobutyl)}-2,2′-bipyridine}ruthenium complex increased. Accordingly, it is proven that addition of 4 μmol of dicetyl phosphate (19.5 mol % relative to the liposome composition constitutive component) resulted in the highest percentage of enclosure, suggesting that approximately more than six times the complex was enclosed in the internal aqueous phase, as compared with the liposome to which dicetyl phosphate was not added. From this result, it is revealed that addition of 4 to 6 μmol of dicetyl phosphate results in capability of achieving inclusion of a larger amount of the bisbipyridine{{4,4′-(4-aminobutyl)}-2,2′-bipyridine}ruthenium complex in the liposome internal aqueous phase.

In addition, it is proven that, the percentage of enclosure of the bisbipyridine{{4,4′-(4-aminobutyl)}-2,2′-bipyridine}ruthenium complex which can be included in the liposome internal aqueous phase is regulated depending on the step wise increase of dicetyl phosphate that imparts a negative charge to the liposome lipid bilayer. Namely, it is concluded that regulation of the negative quantity of electric charge imparted to the lipid bilayer of the liposome enables the percentage of enclosure of the chemical substance included to be arbitrarily regulated.

From the foregoing, it was verified that the liposome internal aqueous phase can include the chemical substance at a high concentration also in Embodiment 3 in which the percentage of enclosure was determined with a measuring method different from the method of Embodiment 1, and that the percentage of enclosure of the chemical substance included can be arbitrarily predetermined by regulating the quantity of electric charge of the lipid bilayer of the liposome.

### Embodiment 4

In Embodiment 4, using the liposome composition prepared in Embodiment 3, measurement of BSA was attempted with a competitive method. In the detection method, electrochemiluminescent detection was executed. Moreover, the liposome composition prepared in Embodiment 3 includes the bisbipyridine{{4,4′-(4-aminobutyl)}-2,2′-bipyridine}ruthenium complex. The structure of this bisbipyridine{{4,4′-(4-aminobutyl)}-2,2′-bipyridine}ruthenium complex has two amines at the linker moiety, and the amines form a tight coordinate bond with the gold electrode. Thus, this complex is likely to be absorbed on the surface of the gold electrode, and the removal thereof is difficult. Therefore, in the competitive reaction accompanied by operations such as disruption and washing of the liposome composition, the bisbipyridine{{4,4′-(4-aminobutyl)}-2,2′-bipyridine}ruthenium complex is suitably used which is likely to be absorbed and hardly removed by these operations.

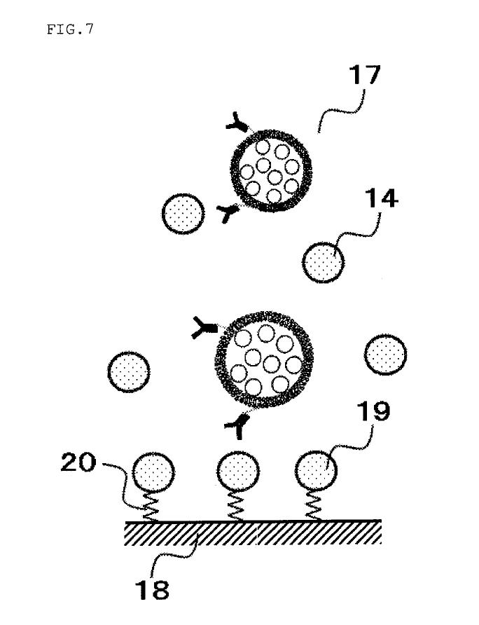

Hereinafter, the competitive method in which the present invention was employed is specifically explained with reference to FIG. 7.

In Embodiment 4, BSA was used as the analyte 14. As the antibody in the liposome 17 modified with the antibody, a BSA antibody was used. In Embodiment 4, the antigen 19 was allowed to bind to the surface of the gold electrode 18 using SAM (Self-Assembled Monolayer) 20. As the antigen 19 immobilized on the gold electrode 18, either the antigen itself, or a peptide having a binding site of the antibody may be used since a competitive method is to be employed.

Hereinafter, Examples in connection with Embodiment 4 are demonstrated.

First, a gold electrode chip provided with a working electrode having a diameter of 3 mm on which an electrode pattern had been formed by a photolithography process on a glass substrate by forming a 200 nm of gold using a sputtering apparatus (manufactured by ULVAC, Inc., SH-350) with 10 nm of titanium as a base was provided. A liquid prepared by mixing sulfuric acid and hydrogen peroxide at a ratio of 3:1 was dropped on the working electrode, and left to stand for 1 min, followed by washing with distilled water, and air drying. Subsequently, 71.5 mg of dithiobutanoic acid (DTBA, 2.52×10⁻⁴ mol) was dissolved in 30 ml of ethanol to prepare a dithiobutanoic acid/ethanol solution. The air dried gold electrode chip was immersed in this solution, and stirred gently at room temperature for 12 hrs to form SAM. After completing the reaction, the resulting SAM was washed with ethanol. After the washing, 50 mg of 1-ethyl-3-(3-dimethylaminopropyl)carbodiimide hydrochloride (2.61×10⁻⁴ mol) and 30 mg of N-hydroxysuccinimide (2.61×10⁻⁴ mol) were dissolved in 10 ml of a 0.1 mol/l phosphate buffer (pH 7.4). The washed gold electrode chip was immersed in this solution, and stirred gently at room temperature for 1 hour to activate carboxyl groups on the working electrode. After this electrode was washed with a 0.1 mol/l phosphate buffer (pH 7.4), it was immersed in a solution prepared by dissolving 1 mg of BSA (4.74×10⁻⁷ mol) in a 10 ml of the 0.1 mol/l phosphate buffer (pH 7.4), and stirred gently at 4° C. for 15 hrs to immobilize the antigen on the electrode surface. After completing the reaction, the gold electrode chip was washed with a 0.1 mol/l phosphate buffer (pH 7.4). Next, 61 mg of 2-aminoethanol (1.0×10⁻³ mol) was dissolved in 10 ml of a 0.1 mol/l phosphate buffer (pH 7.4), and the gold electrode chip was immersed in this solution, followed by gently stirring at 4° C. for 1 hour to permit blocking. After the blocking, the gold electrode chip was equilibrated to an ordinary temperature, and washed with a 0.1 mol/l phosphate buffer (pH 7.4) containing 0.02% Tween 20. Thereafter, the gold electrode chip was air dried, and the area other than the measurement site on the gold electrode chip was covered with a silicon sheet. Onto the gold electrode chip were added each concentration of BSA as the analyte, and the aforementioned liposome composition to allow for a competitive reaction at room temperature for 1 hour. Six different BSA concentrations, i.e., 1.67×10⁻⁷ mol/l, 1.67×10⁻⁸ mol/l, 1.67×10⁻⁹ mol/l, 1.67×10⁻¹⁰ mol/1, 1.67×10⁻¹¹ mol/l and 1.67×10⁻¹² mol/l were employed for the measurement in this process. After completing the competitive reaction, the surface of the gold electrode chip was washed with a 0.1 mol/l phosphate buffer (pH 7.4) containing 0.02% Tween 20, followed by covering with a silicon sheet in a similar to that described above. Thereto was added 7 μl of ethanol to disrupt the liposome composition, and the gold electrode chip was heated at 60° C. for 5 min. After heating, the gold electrode chip was left to stand at room temperature for 5 min, washed with a 0.1 mol/l phosphate buffer (pH 7.4), and air dried. Thereafter, 60 μl of a liquid for electrolytic luminescence containing 0.1 mol/l potassium dihydrogen phosphate, 0.1 mol/l dipotassium hydrogen phosphate and 0.1M TEA was dropped, and subjected to voltage scanning from 0 V to 1.3 V. Thus, the electrochemiluminescence intensity was measured with a photomultiplier tube (manufactured by Hamamatsu Photonics K.K., H7360-01) for 4 sec.

In this measurement, the liposome composition containing 4 μmol of dicetyl phosphate prepared in Embodiment 3 (19.5 mol % relative to the liposome composition constitutive component) was used for conducting the measurement. In addition, for comparing with Conventional Example, a liposome composition to which dicetyl phosphate was not added (liposome composition prepared without adding dicetyl phosphate in Embodiment 3) was subjected to a similar measurement to carry out the comparison.

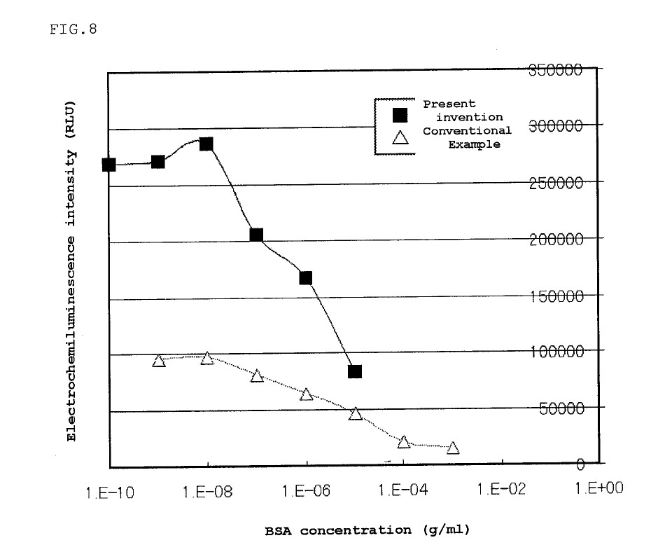

The measurement results are shown in FIG. 8.

As is shown in FIG. 8, when the present invention is compared with Conventional Example at the same BSA concentrations, it was found that the present invention achieved 2 to 0.3 times the electrochemiluminescence intensity. Namely, since very enhanced luminescence strength is attained by using the liposome composition of the present invention, it can be concluded that measurement with high sensitivity is enabled even though a convenient detection apparatus is used.

As described hereinabove, the results of Embodiments 1 to 4 demonstrate that the liposome composition of the present invention can include a high concentration of a chemical substance in the liposome internal aqueous phase, and thus an intense luminescent signal can be generated with a high sensitivity by using this liposome composition as a labelling agent for immunoassay technique and the like.

### INDUSTRIAL APPLICABILITY

The liposome composition and the production method thereof, and the analytical method of an analyte using the same according to the present invention enable a chemical substance to be included at a high concentration by imparting a positive or negative charge to the liposome lipid bilayer, and including the chemical substance having a charge opposite to the charge of the liposome lipid bilayer in the liposome internal aqueous phase. Thus, inclusion of a chemical substance that generates a signal such as an electrochemiluminescent substance or a chemiluminescent substance enables an analyte to be measured with a high sensitivity, it is useful in food analysis and clinical inspection fields, and the like. Namely, an analytical method capable of generating an intense luminescent signal with a high sensitivity can be provided, leading to simplification of the detection apparatus, and prospection for, for example, size reduction of point of care testing devices in clinical inspection fields. In addition, by enabling a high concentration of a chemical substance to be included in a liposome internal aqueous phase, the amount of inclusion of the chemical substance can be freely selected; therefore, applications to drug delivery systems anticipated for enhancement of the effects and alleviation of side effects of medical drugs, as well as applications to cosmetics including a pharmaceutical preparation can be also expected.

What is claimed is:

1. A liposome composition comprising a liposome, and a chemical substance enclosed in an internal aqueous phase of the liposome, wherein:
a lipid bilayer composing the liposome has a positive or negative charge; and
the chemical substance has a charge opposite to the charge of the lipid bilayer.

2. The liposome composition according to claim 1, wherein the percentage of enclosure of the chemical substance included in the internal aqueous phase is adjusted depending on the quantity of electric charge of the lipid bilayer in the liposome composition.

3. The liposome composition according to claim 1 or 2, wherein the component that forms the lipid bilayer comprises either one or both of a phospholipid and a glycolipid.

4. The liposome composition according to any one of claims 1 to 3, wherein the liposome has a ligand on the external surface.

5. The liposome composition according to claim 4, wherein the ligand is at least one selected from the group consisting of an antibody, an antigen, a peptide, a nucleic acid, protein A, protein G, avidin, and biotin.

6. The liposome composition according to any one of claims 1 to 5, wherein the liposome is a monolayer liposome.

7. The liposome composition according to claim 6, wherein the monolayer liposome has a diameter falling within the range of 20 to 200 nm.

8. The liposome composition according to any one of claims 1 to 7, wherein the chemical substance is hydrophilic.

9. The liposome composition according to any one of claims 1 to 8, wherein the component that forms the lipid bilayer comprises a lipid bilayer component having a negative charge, and the lipid bilayer component having a negative charge imparts a negative charge to the lipid bilayer.

10. The liposome composition according to claim 9, wherein the lipid bilayer component having a negative charge comprises at least one selected from the group consisting of an acidic phospholipid, a glycolipid having sialic acid, and a phosphoric acid ester.

11. The liposome composition according to claim 10, wherein the phosphoric acid ester is dicetyl phosphate.

12. The liposome composition according to any one of claims 1 to 11, wherein the chemical substance is a chemical substance having a positive charge.

13. The liposome composition according to claim 12, wherein the chemical substance having a positive charge is a metal complex.

14. The liposome composition according to claim 13, wherein the metal complex has an electrochemiluminescent property.

15. The liposome composition according to claim 13 or 14, wherein the metal complex comprises as a ligand at least one selected from the group consisting of a heterocyclic compound, and an organic compound containing at least one functional group selected from the group consisting of an amino group, a phosphino group, a carboxyl group and a thiol group.

16. The liposome composition according to claim 15, wherein the heterocyclic compound is a compound having a pyridine moiety.

17. The liposome composition according to claim 16, wherein the compound having a pyridine moiety is a compound having a bipyridine skeleton or a phenanthroline skeleton.

18. The liposome composition according to claim 15, wherein the organic compound is bis(diphenylphosphino)ethene.

19. The liposome composition according to any one of claims 13 to 18, wherein the metal complex contains ruthenium or osmium as a central metal.

20. The liposome composition according to any one of claims 1 to 8, wherein the component that forms the lipid bilayer comprises a lipid bilayer component having a positive charge, and the lipid bilayer component having a positive charge imparts a positive charge to the lipid bilayer.

21. The liposome composition according to claim 20, wherein the lipid bilayer component having a positive charge is an amine compound.

22. The liposome composition according to claim 21, wherein the amine compound is stearylamine.

23. The liposome composition according to any one of claims 20 to 22, wherein the chemical substance is a chemical substance having a negative charge.

24. A method for producing a liposome composition comprising the step of enclosing a chemical substance in the internal aqueous phase of the liposome by forming the liposome in the presence of at least one of a phospholipid and a glycolipid, a lipid bilayer-forming component that imparts a positive or negative charge to a lipid bilayer composing the liposome, a chemical substance having a charge opposite to the charge of the lipid bilayer, and water.

25. The method for producing a liposome composition according to claim 24 comprising the steps of:
forming a lipid film comprising at least one of a phospholipid and a glycolipid, and a lipid bilayer-forming component that imparts a positive or negative charge to a lipid bilayer composing the liposome;
bringing the lipid film into contact with an aqueous solution dissolving a chemical substance having a charge opposite to the charge of the lipid bilayer-forming component; and
forming the liposome including the chemical substance by mixing the lipid film with the aqueous solution, after the step of bringing the lipid film into contact with the aqueous solution.

26. The method for producing a liposome composition according to claim 24 or 25 further comprising the step of modifying with a ligand the external surface of the liposome formed in the step of enclosing.

27. The method for producing a liposome composition according to any one of claims 24 to 26, wherein the component that imparts a negative charge to the lipid bilayer comprises at least one selected from the group consisting of an acidic phospholipid, a glycolipid having sialic acid, and a phosphoric acid ester.

28. The method for producing a liposome composition according to any one of claims 24 to 27, wherein the chemical substance is a chemical substance having a positive charge.

29. The method for producing a liposome composition according to any one of claims 24 to 26, wherein the component that imparts a positive charge to the lipid bilayer comprises stearylamine.

30. The method for producing a liposome composition according to any one of claims 24 to 26 and 29, wherein the chemical substance is a chemical substance having a negative charge.

31. An analytical method of an analyte comprising
analyzing the analyte with a sandwich method or a competitive method using the liposome composition according to claim 4 by way of a binding reaction between the ligand and the analyte,
the chemical substance being capable of generating a signal.

32. The analytical method of an analyte carried out using the liposome composition according to claim 31, wherein the chemical substance having a positive charge is a metal complex.

33. The analytical method of an analyte carried out using the liposome composition according to claim 32, wherein the metal complex has an electrochemiluminescent property or a fluorescence property.

34. The analytical method of an analyte carried out using the liposome composition according to claim 32 or 33, wherein the metal complex comprises as a ligand at least one selected from the group consisting of a heterocyclic compound, and an organic compound containing at least one functional group selected from the group consisting of an amino group, a phosphino group, a carboxyl group and a thiol group.

35. The analytical method of an analyte carried out using the liposome composition according to claim 34, wherein the heterocyclic compound is a compound having a pyridine moiety.

36. The analytical method of an analyte carried out using the liposome composition according to claim 35, wherein the compound having a pyridine moiety is a compound having a bipyridine skeleton or a phenanthroline skeleton.

37. The analytical method of an analyte carried out using the liposome composition according to claim 34, wherein the organic compound is bis(diphenylphosphino)ethene.

38. The analytical method of an analyte carried out using the liposome composition according to any one of claims 32 to 37, wherein the metal complex contains ruthenium or osmium as a central metal.

39. The analytical method of an analyte carried out using the liposome composition according to any one of claims 31 to 38, wherein the signal is an electrochemiluminescence or fluorescence.

40. The analytical method of an analyte carried out using the liposome composition according to any one of claims 31 to 39, wherein the liposome is a monolayer liposome.

41. The analytical method of an analyte carried out using the liposome composition according to claim 40, wherein the monolayer liposome has a diameter falling within the range of 20 to 200 nm.

42. The analytical method of an analyte carried out using the liposome composition according to any one of claims 31 to 41, wherein the ligand is at least one selected from the group consisting of an antibody, an antigen, a peptide, a nucleic acid, protein A, protein G, avidin, and biotin.

43. The analytical method of an analyte carried out using the liposome composition according to any one of claims 31 to 42, wherein the analyte is at least one selected from the group consisting of an antibody, an antigen, a peptide, and a nucleic acid.
